(19) United States
(12) Patent Application Publication
(10) Pub. No.: US 2021/0177477 A1
(43) Pub. Date: Jun. 17, 2021

(54) CANNULATED ASPIRATION SCREW DEVICE AND METHODS OF USE THEREOF

(71) Applicant: Alliance Partners LLC, San Antonio, TX (US)

(72) Inventors: Anish Vaghela, Boerne, TX (US); Brad Fredin, Buda, TX (US); Andrew F. Cannestra, Jacksonville, FL (US)

(73) Assignee: Alliance Partners LLC, San Antonio, TX (US)

(21) Appl. No.: 17/120,568

(22) Filed: Dec. 14, 2020

Related U.S. Application Data
(60) Provisional application No. 62/948,053, filed on Dec. 13, 2019.

(51) Int. Cl. A61B 17/86

(52) U.S. Cl. CPC A61B 17/864; A61B 2217/005; A61B 17/8635

(57) ABSTRACT

An improved cannulated aspiration screw device and method of use thereof for removal of, inter alia, bone marrow from a patient's bone. The cannulated aspiration screw device includes a shaft at its proximate end and a drilling dip section at its distal end and has a removable insert, that when inserted in the cannulated aspiration screw device at is proximate end prevents the flow of materials into and through the cannulated aspiration screw device while the device is being screwed into the bone. Once the cannulated aspiration screw device is screwed in place, the removable insert is removed, and an aspiration device (such as a surgical syringe) is then connected at the proximate end of the cannulated aspiration screw device (generally utilizing a connector) and aspiration is then performed using the aspiration device.

### CROSS-REFERENCE TO RELATED PATENTS/APPLICATIONS

This application claims priority benefits to U.S. Patent Application Serial No. 62/948,053, entitled “Cannulated Aspiration Screw Device And Methods of Use Thereof,” filed on Dec. 13, 2019. This patent application is commonly assigned to the Assignee of the present invention and is hereby incorporated herein by reference in its entirety for all purposes.

### FIELD OF INVENTION

An aspiration device and method of use thereof, and more particularly a cannulated aspiration screw device and method of use thereof for removal of, inter alia, bone marrow from a patient's bone.

### BACKGROUND OF INVENTION

Bone is made up of a hard outer core, known as cortical bone or cortical plate, and a soft spongy interior known as cancellous bone or trabecular bone, which includes a marrow filling in the porous space within the spongy bone (commonly referred to as bone marrow). The cortical plate is very hard and provides the rigid structure to the skeleton, which allows the skeleton to bear weight. Bone marrow is rich in capillary beds.

Bone marrow is can be utilized in orthopedic procedures to augment fracture healing for a patient. Bone marrow is also an excellent source of mesenchymal stem cells (MSC's) and/or tissue progenitor cells (TPC's). Moreover, these multi-potent cells have broad applications in addition to orthopedics and may be used in the fields of cardiology, oncology and other areas. As new techniques are being developed to use these cells and to culture them ex-vivo, it has become important to be able to procure large volumes of highly cellular marrow from the patient's body.

Generally, the patient's own bone marrow is utilized for procedures on the patient, and typically, the pelvis (i.e., hip bone, including, particularly, the iliac bone) is generally the primary source for the marrow that is utilized. Traditional bone marrow aspiration needle are used to access marrow from the hip or iliac bone. The traditional aspiration needle (such as shown in U.S. Pat. No. 5,807,275, issued Sep. 15, 1998 to K. Jamshidi) includes an aspiration cannula (also referred to as a cannulated trocar) and a removable stylet that extends through the cannula and has a sharp tip. Once the needle is through the cortical plate, the cannula has access to the bone marrow that is located around the cannula tip. Harvesting marrow, however, can be difficult, particularly in view of the hard outer core of the bone (and especially the larger pelvis bone). Moreover, once the outer core is penetrated, there is a need to smoothly transition between the steps of insertion of the device to being able to utilize the device for aspiration and vice versa (when the device is to be removed).

Accordingly, there remains a need for an improved aspiration device.

### SUMMARY OF INVENTION

The present invention regards an improved cannulated aspiration screw device and method of use thereof for removal of, inter alia, bone marrow from a patient's bone.

In general, in one aspect, the invention features a cannulated aspiration screw device having a longitudinal axis. The cannulated aspiration screw device includes a shaft having a first end and a second end. The first end of the shaft has a first tubular section along the longitudinal axis that is substantially cylindrical and having a first outer-diameter. The second end of the shaft has a second tubular section along the longitudinal axis that is substantially cylindrical and having a second outer-diameter. The second outer-diameter is smaller than the first outer-diameter. The cannulated aspiration screw device further includes a drilling tip section having a first end and a second end. The first end of the drilling tip section is connected to the first end of the shaft and positioned along the longitudinal axis. The second end of the drilling tip section has a drilling tip that is operable for drilling into bone. The drilling tip includes one or more fenestrations. The shaft and drilling tip section are operable for receiving a removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft. In the absence of the removable insert, the cannulated aspiration screw device is operable for flowing materials through the one or more fenestrations into the drilling tip section, from the drilling tip section to the shaft, and from the shaft to outside the shaft through the second end of the shaft.

Implementations of the invention can include one or more of the following features:

The shaft and drilling tip section can include biocompatible transient contact only materials.

The one or more fenestrations can include at least two fenestrations.

The one or more fenestrations can have a diameter that is at most a 1:1 ratio of fenestration diameter to pitch.

The removable insert can be inserted in the cannulated aspiration screw device and the removable insert can prevent the flow of materials through the one or more fenestrations, from the drilling tip section to the shaft, and out of the shaft through the second end of the shaft.

The removable insert can be a stylet.

The removable insert can seat on the one or more fenestrations.

The cannulated aspiration screw device can be operatively connected to a manual drill or handle.

The removable insert can be not inserted in the cannulated aspiration screw device.

The cannulated aspiration screw device can be sealably connected to an aspiration device.

The cannulated aspiration screw device can be sealably connected to an aspiration device with a connector.

The connector can be a male luer connector.

The connector can include an O-ring and a positive stop.

The aspiration device can be a syringe.

The syringe can be a standard surgical syringe.

In general, in another aspect, the invention features an aspiration method that includes selecting a cannulated aspiration screw device including a shaft and a drilling tip section. The drilling tip section includes one or more fenestrations. The cannulated aspiration screw device has a removable insert that prevents materials from flowing into the cannulated aspiration screw device through the one or more fenestrations. The aspiration method further includes screwing the drilling tip section of the cannulated aspiration screw device into a bone containing bone marrow. The aspiration method further includes removing the removable insert from the cannulated aspiration screw device after the step of screwing the drilling tip section in the bone. The aspiration method further includes connecting the cannulated aspiration screw device to an aspiration device. The aspiration method further includes utilizing the aspiration device to aspirate bone marrow from the bone through the cannulated aspiration screw device. The aspiration method further includes removing the cannulated aspiration screw device from the bone.

Implementations of the invention can include one or more of the following features:

The cannulated aspiration screw device can be one of the above-described cannulated aspiration screw devices.

The shaft of the cannulated aspiration screw device can have a first end and a second end. The first end of the shaft can have a first tubular section along the longitudinal axis that is substantially cylindrical and having a first outer-diameter. The second end of the shaft can have a second tubular section along the longitudinal axis that is substantially cylindrical and having a second outer-diameter. The second outer-diameter can be smaller than the first outer-diameter. The drilling tip section of the cannulated aspiration screw device can have a first end and a second end. The first end of the drilling tip section can be connected to the first end of the shaft and positioned along the longitudinal axis. The second end of the drilling tip section can have a drilling tip that is operable for drilling into bone. The shaft and drilling tip section can be operable for receiving the removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft. In the absence of the removable insert, the cannulated aspiration screw device can be operable for flowing materials through the one or more fenestrations into the drilling tip section, from the drilling tip section to the shaft, and from the shaft to outside the shaft through the second end of the shaft.

The step of screwing the drilling tip section of the cannulated aspiration screw device into the bone containing bone marrow can include connecting a proximate end of the shaft to a manual drill or handle and can include using the manual drill or handle to screw the cannulated aspiration screw device.

The manual drill or handle can be a manual drill.

The manual drill or handle can be connected to the proximate end of the shaft without any connector.

The manual drill or handle can be connected to the proximate end of the shaft with a connector that connects to both the manual drill or handle and the proximate end of the shaft.

The step of connecting the cannulated aspiration screw device to an aspiration device can include connecting a connector to the proximate end of the shaft and connecting the connector to the aspiration device.

The connector can be a male luer connector.

The connector can include an O-ring and a positive stop.

The aspiration device can be a syringe.

The syringe can be a standard surgical syringe.

The aspiration method can further include removing the connector from the cannulated aspiration screw device.

The step of removing the cannulated aspiration screw device from the bone can include connecting a proximate end of the shaft to a manual drill or handle and can include using the manual drill or handle to unscrew the cannulated aspiration screw device.

The manual drill or handle can be a manual drill.

In general, in another aspect, the invention features a kit that includes a plurality of components. The plurality of components include a cannulated aspiration screw device including a proximate end and a distal end. The proximate end of the cannulated aspiration screw device comprises a shaft. The distal end of the cannulated aspiration screw device includes a drilling tip section. The drilling tip section includes one or more fenestrations. The cannulated aspiration screw device is operable to be screwed into bone using the drilling tip section. The plurality of components further include a removable insert that is operable to be inserted into the proximate end of the cannulated aspiration screw device and through the shaft and drilling tip section to prevents materials from flowing into the cannulated aspiration screw device through the one or more fenestrations. The plurality of components further include a connector that is operable to sealably connect the proximate end of the cannulated aspiration screw device to an aspiration device.

Implementations of the invention can include one or more of the following features:

The cannulated aspiration screw device can be one of the above-described cannulated aspiration screw devices.

The shaft of the cannulated aspiration screw device can have a first end and a second end. The first end of the shaft can have a first tubular section along the longitudinal axis that is substantially cylindrical and having a first outer-diameter. The second end of the shaft can have a second tubular section along the longitudinal axis that is substantially cylindrical and having a second outer-diameter. The second outer-diameter can be smaller than the first outer-diameter. The drilling tip section of the cannulated aspiration screw device can have a first end and a second end. The first end of the drilling tip section can be connected to the first end of the shaft and positioned along the longitudinal axis. The second end of the drilling tip section can have a drilling tip that is operable for drilling into bone. The shaft and drilling tip section can be operable for receiving the removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft. In the absence of the removable insert, the cannulated aspiration screw device can be operable for flowing materials through the one or more fenestrations into the drilling tip section, from the drilling tip section to the shaft, and from the shaft to outside the shaft through the second end of the shaft.

The cannulated aspiration screw device can include biocompatible transient contact only materials.

The plurality of components can be stabilizable.

The aspiration device can be a surgical syringe.

The plurality of components can further include the aspiration device.

The aspiration device can be a surgical syringe.

The connector can be a male luer connector.

The connector can include an O-ring and a positive stop.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 depicts a perspective view of a cannulated aspiration screw device (with inserted stylet) of the present invention.

FIG. 2 depicts an exploded view of the cannulated aspiration screw device (including stylet) shown in FIG. 1.

FIGS. 3A-3D depict the drilling tip section of the cannulated aspiration screw device of FIG. 1 that is located at the distal end of the cannulated aspiration screw device. FIGS. 3A-3D depict, respectively, the side, distal, proximate, and perspective views of the drilling tip section.

FIGS. 4A-4C depict the shaft section of the cannulated aspiration screw device of FIG. 1. FIGS. 4A-4C depict, respectively, the perspective, distal, and proximate views of the shaft section.

FIGS. 5A-5B depict the stylet for the cannulated aspiration screw device of FIG. 1. FIGS. 5A-5B depict, respectively, the perspective and distal/proximate views of the stylet.

FIG. 6 depicts a perspective view of the cannulated aspiration screw device of FIG. 1 after it has had the stylet removed and a connector has been attached at the proximate end of the cannulated aspiration screw device.

FIGS. 7A-7C depict the connector shown in FIG. 6. FIGS. 7A-7C depict, respectively, the side, distal, and proximate views of the connector.

FIG. 8A depicts the side view of the connector shown in FIG. 6 with arrows showing a direction of view of A-A′.

FIG. 8B depicts a cross-section of the connector shown in FIG. 6 taken from view A-A′ shown in FIG. 8A.

FIG. 9A depicts a side view the cannulated aspiration screw device of FIG. 6 connected to a syringe for aspiration.

FIG. 9B depicts a magnified portion of FIG. 9A.

FIG. 10 is a flowchart of a surgical technique utilizing the cannulated aspiration screw device of the present invention.

### DETAILED DESCRIPTION

A cannulated aspiration screw device and method of use thereof for removal of, inter alia, bone marrow from a patient's bone. The cannulated aspiration screw device can be used with a removable stylet and an attachable connector (such as a luer connector). The stylet is utilized within the cannulated aspiration screw device during inserting into the bone (to prevent bone chips from entering the cannulated screw. The luer connector (generally a male luer connector) is used during the aspiration of the marrow, including to connect to a standard surgical syringe (or other aspiration device) for aspiration. These components of the device and its various stylet and attachments are generally made of biocompatible transient contact only materials. Indeed, these can be arranged in a kit (either a sterilized kit or one that gas components that can be sterilized on-site, such as at the hospital) containing the device, stylet, luer connector, and/or other connectors. The cannulated aspiration screw device is intended for use for aspiration of bone marrow or autologous blood by use of a standard surgical syringe (or other aspiration device).

### Cannulated Aspiration Screw Devices

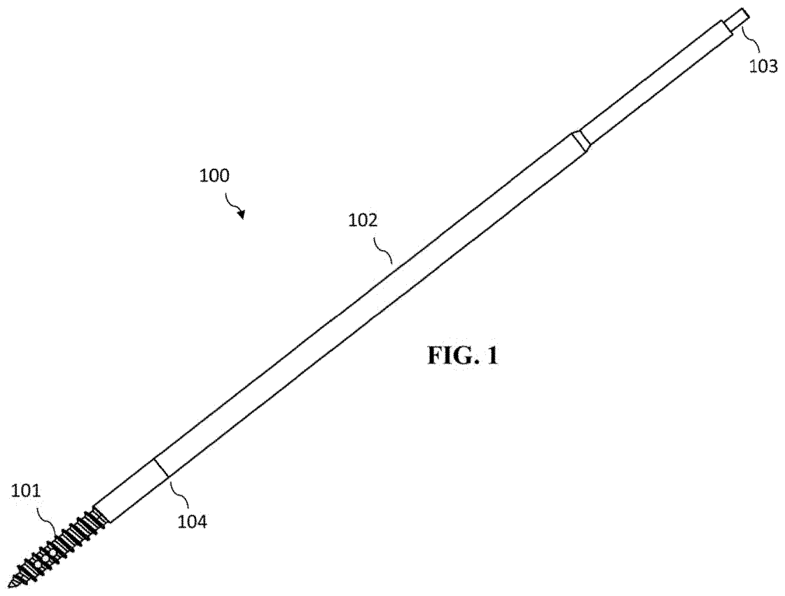

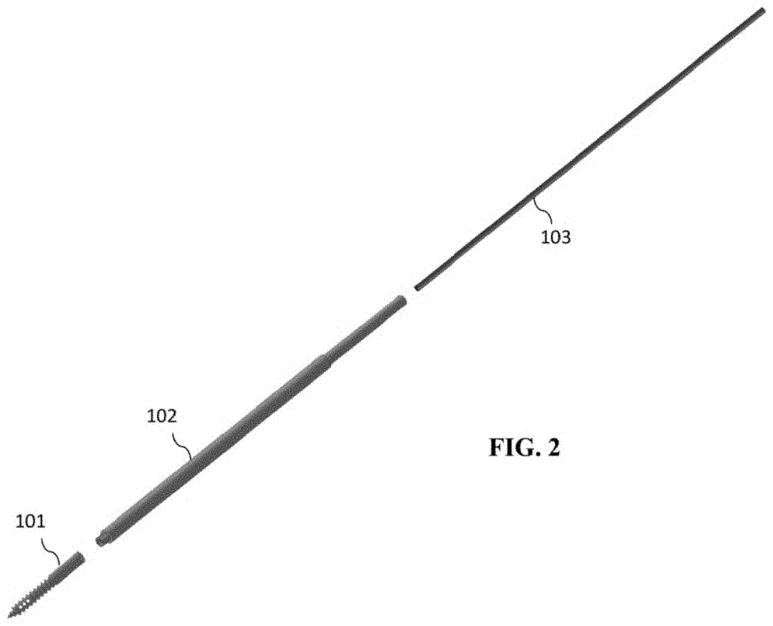

Referring now to the figures, FIG. 1 shows a perspective view of a cannulated aspiration screw device 100 (with inserted stylet 103). FIG. 2 shows an exploded view of the cannulated aspiration screw device 100 (which includes a drilling tip 101 and a shaft 102) and stylet 103. The self-drilling tip 101 and the shaft 102 couple to one another with a lap joint, which can be welded.

For orientation purposes, (a) the end of shaft 102 through which the stylet 103 is inserted is referred to as the “proximate” end of cannulated aspiration screw device 100 and (b) drilling tip 101 is located at the end of cannulated aspiration screw device 100 and drilling tip 101 that is referred to as the “distal” end of cannulated aspiration screw device 100. Such orientation of “proximate” and “distal” will be maintained relative to the drilling tip 101 and shaft 102 (i.e., the distal end of shaft 102 is connected to the proximate end of drilling tip 101).

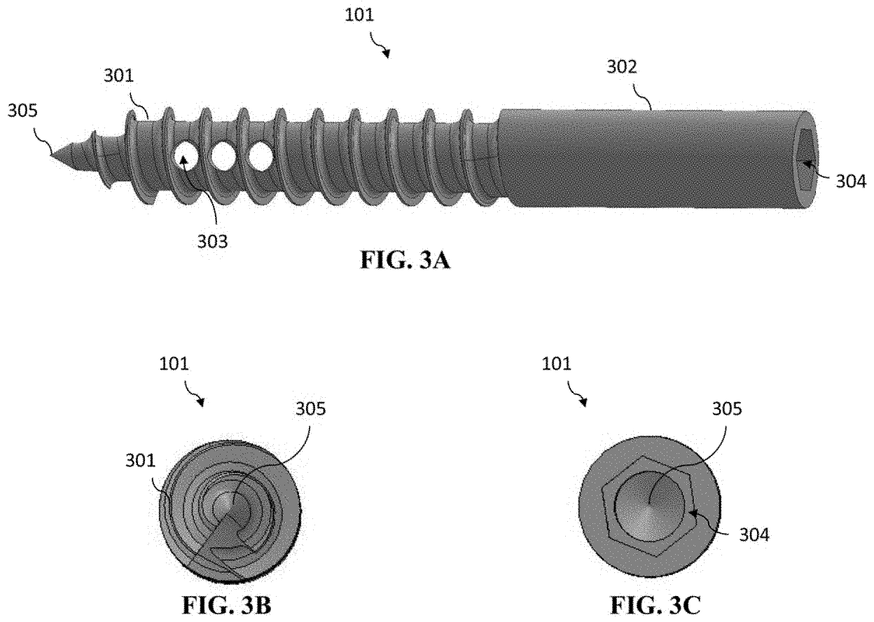

FIGS. 3A-3D shows the drilling tip 101 (which, as shown, is a self-drilling tip) of the cannulated aspiration screw device 100. FIGS. 3A-3D show, respectively, the side, distal, proximate, and perspective views of the drilling tip 101. The proximate end 302 of drilling tip 101 has a shaped structure 304 that can be coupled to the distal end 301 of shaft 102. Distal end 301 is a self-drilling threaded tip section and includes tip 305 located at the furthermost distal end of drilling tip 101. For orientation purposes, the “distal view” is the longitudinal view taken from the distal end, i.e., the distal view of self-drilling tip 101 is a view looking at the tip 305 along the longitudinal axis of the drilling tip 101. Drilling tip 101 and its self-drilling threaded tip allow for the cannulated aspiration screw device 100 to be inserted into bone (such as by rotating the cannulated aspiration screw device with a drill).

Drilling tip 101 has fenestrations 303. It is though fenestrations 303 that bone marrow (or other materials) can be aspirated into the cannulated aspiration screw device 100. The interior of drilling tip 101 is hollow so that the materials can flow into the fenestrations 303 and transverse in the interior of drilling tip 101 along its longitudinal axis o and out through proximate end 302 of the drilling tip 101. For orientation purposes, the “proximate view” of is the longitudinal view taken from the proximate end, i.e., the proximate view of drilling tip 101 is a view looking at proximate end 302 along the longitudinal axis of drilling tip 101. Because the interior of drilling tip 101 is hollow, the back side of tip 305 (located at the farthermost distal end of drilling tip 101) can still be seen in the proximate view shown FIG. 3C. As shown in FIGS. 3A and 3D, there can be three fenestrations 303 on either side of drilling tip 101. Generally, drilling tip 101 should have at least two fenestrations transverse to the longitudinal bore of the cannulated aspiration screw device 100, with the fenestrations located along the screw minor. The fenestrations should be at most in a 1:1 ratio of fenestration diameter to pitch.

Fenestrations 303 are situation such that, when stylet 103 is in cannulated aspiration screw device 100, fenestrations 303 are closed in that the stylet 103 is seated on the fenestrations and prevents the flow of materials through fenestrations 303.

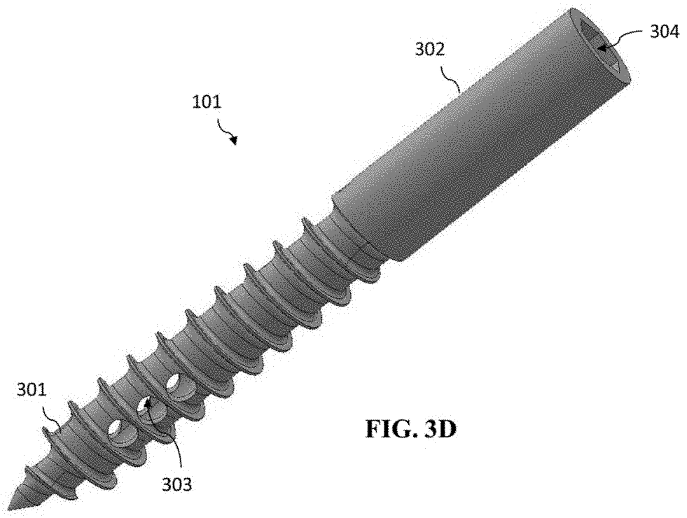

FIGS. 4A-4C shows the shaft 102 of the cannulated aspiration screw device 100. FIGS. 4A-4C show, respectively, the perspective, distal, and proximate views of the shaft 102. The shaft 102 has a generally uniform diameter elongated shaft section 401 that has a shaped structure 403 at the distal end of shaft 102 that can couple with shaped structure 304 of drilling tip 101. After coupling of shaped structures 304 and 403 (at the lap joint), the interface can be weld together.

Shaft 102 can have a proximate end that has a smaller outer-diameter section 402. Shaft 102 is cylindrical in that it is hollow running in a longitudinal direction. At the proximate end of section 402 is a hole 404 through which the stylet 103 can be inserted into cannulated aspiration screw device 100. Section 402 can be connected to a connector for various purposes, such as during aspiration (as discussed below). Section 402 can also be connected to a connector for attachment to a drill or handle (not shown) that can rotate the cannulated aspiration screw device 100 when inserting to bone. In some embodiments, section 402 can be directly connected to the drill/handle without the need (or use) of a connector.

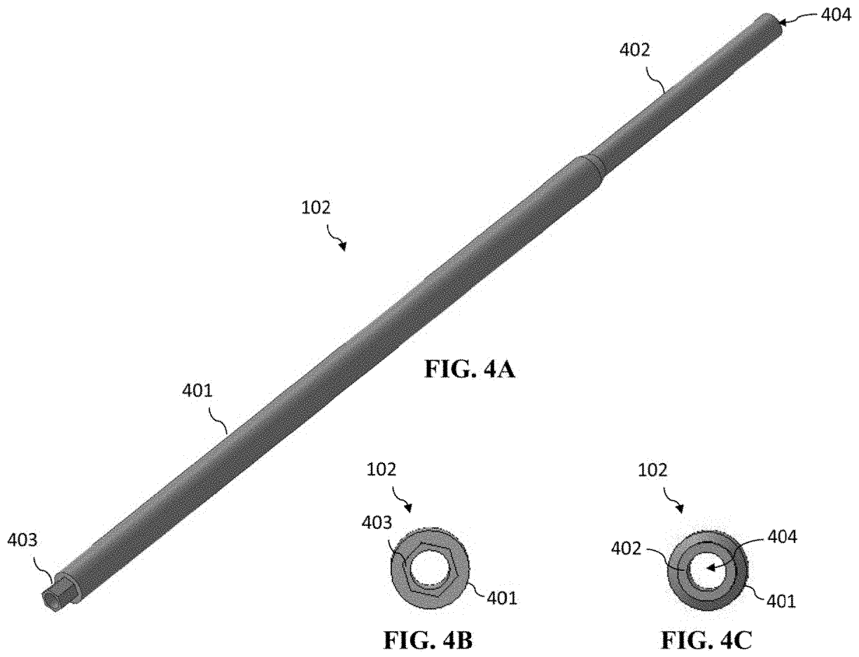

FIGS. 5A-5B show stylet 103 for cannulated aspiration screw device 100. FIGS. 5A-5B depict, respectively, the perspective and distal/proximate views of stylet 103. Stylet 103 can be inserted into cannulated aspiration screw device 100 by inserting stylet 103 through hole 404, which then runs longitudinally along the interior hollow sections of shaft 102 and drilling tip 101. As noted above, once the stylet is set in this position, materials cannot flow into the cannulated aspiration screw device 100 through fenestrations 303 (i.e., fenestrations 303 are closed. Because stylet 103 is symmetrical, the distal and proximate views are the same (and thus both are shown in FIG. 5B). Optionally, the distal end of stylet 103 can include a small projecting knob, ridge, or other knurl for better grip.

Stylet 103 is inserted into cannulated aspiration screw device 100 while cannulated aspiration screw device 100 is being inserted into the hip bone or other bone. Again, this is to prevent bone chips from entering cannulated aspiration screw device 100. Once cannulated aspiration screw device 100 is in place, the stylet is removed and a connector can be attached for attaching the aspiration device (such as a syringe).

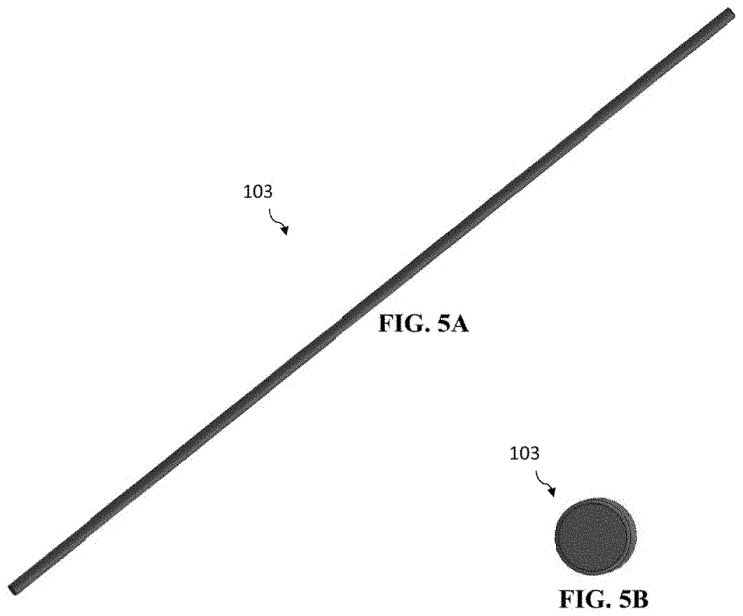

FIG. 6 shows a perspective view of the cannulated aspiration screw device 100 after it has had the stylet 103 removed and a connector 601 (also called a detachable adapter) has been attached at the proximate end of the cannulated aspiration screw device 100. Connector 601 can be a male luer connector that can operatively connect to the aspiration device. Optionally, the connector 601 can include a small projecting knob, ridge, or other knurl for a better grip.

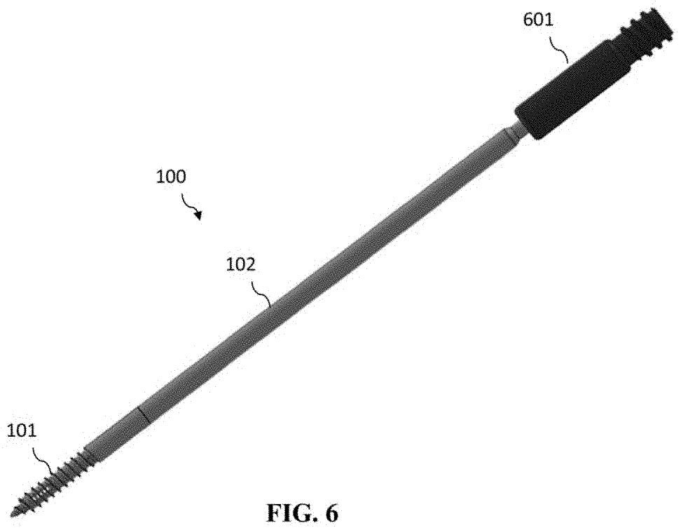

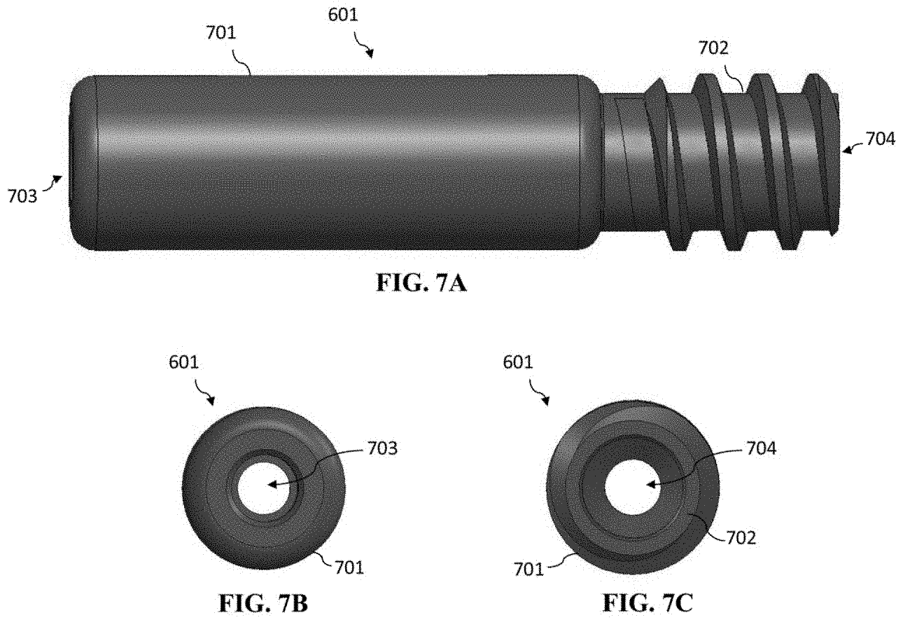

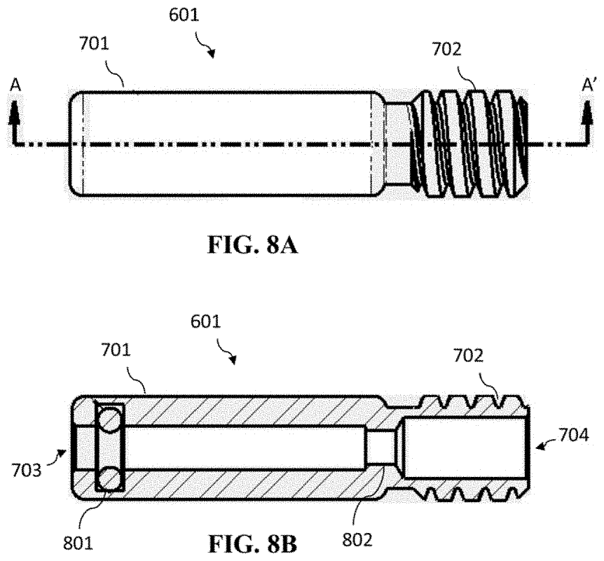

FIGS. 7A-7C and 8A-8B shows the connector 601, with FIGS. 7A-7C showing, respectively, the side, distal, and proximate views of connector 601, FIG. 8A showing the side view of the connector 601 (with arrows showing a direction of view of A-A′), and FIG. 8B showing a cross-section of the connector 601 taken from view A-A′. Connector 601 has a distal end 701 that can be coupled to smaller outer-diameter section 402. Smaller outer-diameter section 402 can inserted into distal end 701 of connector 601 through hole 703. These can then be coupled and sealed utilizing O-rings 801. There is a positive stop 802 in connector 601 that prevents smaller outer-diameter section 402 from being over inserted into connector 602.

The proximate end 702 is threaded so it can be coupled to the surgical syringe or other aspiration device. The materials can then be aspirated from the cannulated aspiration screw device 100 through the connector 602 and out hole 704 into a receptacle of the aspiration device (such as the cylindrical tube of a syringe).

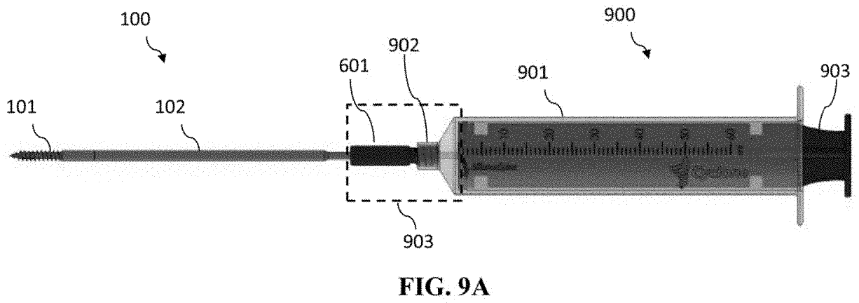

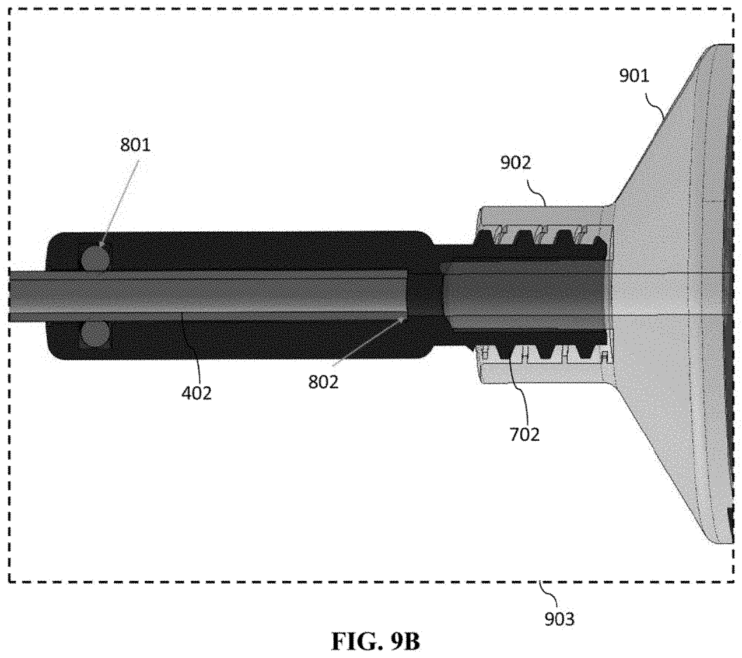

FIG. 9A shows a side view the cannulated aspiration screw device 100 connected using connector 601 to a syringe 900 for aspiration. FIG. 9B shows (in magnification) box 903 of FIG. 9A. Smaller outer-diameter section 402 has been inserted into distal end 701 of connector 601 and then coupled and sealed utilizing O-rings 801. A syringe 900 (such as a 60 ml syringe) has been engaged with the connector 601 (which thus engages syringe 900 with cannulated aspiration screw device 100). Proximate end 702 of connector 601 sealably couple with syringe 900 with the threads on tip 902 of syringe 900. Plunger 903 can then be move to aspirate materials (such as bone marrow) into syringe cylinder 901. The bone marrow aspirated materials can then be used directly (by injecting from the syringe) or moved to another receptacle for storage and use.

### Methods Using Cannulated Aspiration Screw Devices

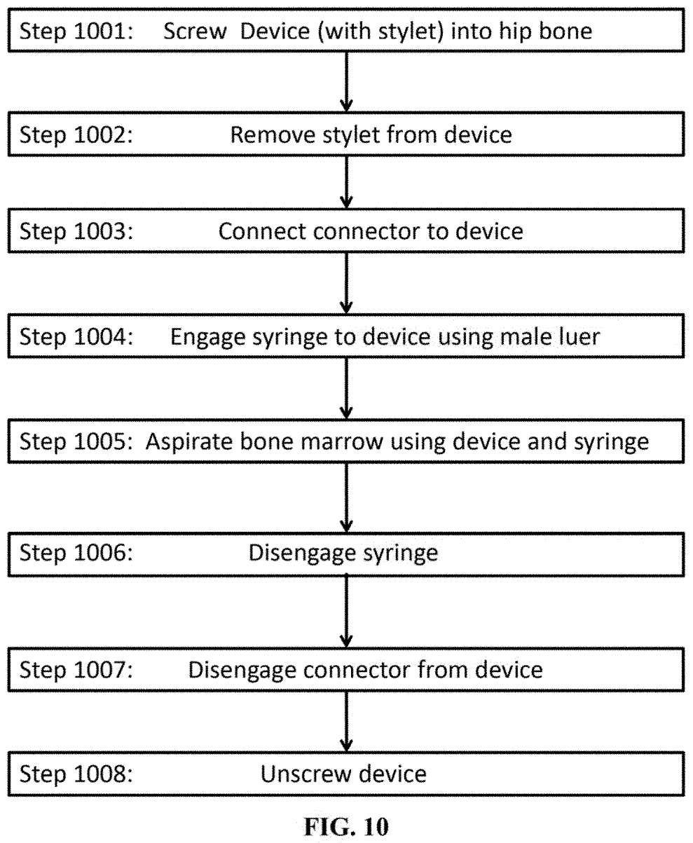

FIG. 10 is a flowchart of a surgical technique using a cannulated aspiration screw device, such as cannulated aspiration screw device 100. A surgeon or other practitioner can use the cannulated aspiration screw device of the present invention in a method that includes the following steps:

In step 1001, the surgeon/practitioner screws the cannulated aspiration screw device 100 (with the stylet 103 inserted) into the hip bone (or other target bone). This can be done using a drill/handle that is connected to section 402 of shaft 102 directly to the drill/handle or by using a drill/handle connector that connects section 402 to the drill/handle. The rotation of the drill/handle then screws the cannulated aspiration screw device into the hip bone. The drill/handle can be, for example, a manual drill (such as a hand drill), a power drill, or a handle.

Once the cannulated aspiration screw device is screwed into the bone, in step 1002, the surgeon/practitioner can remove the stylet. In this step, the surgeon/practitioner can also detach the drill/handle or other device used to rotate the cannulated aspiration screw device.

In step 1003, the surgeon/practitioner connects a connector to the proximate end of the cannulated aspiration screw device. This can be a male luer connector or other adaptor that is slid onto the proximate end and then sealably connected.

In step 1004, the surgeon/practitioner then engages the syringe (or other aspiration device) to the connector (which thereby engages the syringe to the cannulated aspiration screw device.

In step 1005, the surgeon/practitioner then utilizes the syringe to aspirate bone marrow from the hip bone, into the cannulated aspiration screw device through the fenestrations, through the cannulated aspiration screw device, and into the cylinder of the syringe.

Once aspiration is complete, in step 1006, the surgeon/practitioner can then disengage the syringe from the connector and the cannulated aspiration screw device.

In step 1007, the surgeon/practitioner can then disengage the connector from the device.

In step 1008, the surgeon/practitioner can then unscrew the device. Optionally, the surgeon/practitioner can do so using a drill/handle to rotate the cannulated aspiration screw device in the direction that would unscrew the device.

A medical procedure kit (or set) can be utilized that fully supports the surgical procedure set forth in FIG. 10. The kit can include the cannulated aspiration screw device as well as one or more of the following, a stylet, a connector, and a syringe. Generally, the syringe is a standard surgical syringe and thus this could be used in lieu of having one in the kit.

The combination of tools in the kit can be pre-sterilized for ready use.

The disclosures of all patents, patent applications, and publications cited herein are hereby incorporated herein by reference in their entirety, to the extent that they provide exemplary, procedural, or other details supplementary to those set forth herein. It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of approximately 1 to approximately 4.5 should be interpreted to include not only the explicitly recited limits of 1 to approximately 4.5, but also to include individual numerals such as 2, 3, 4, and sub-ranges such as 1 to 3, 2 to 4, etc. The same principle applies to ranges reciting only one numerical value, such as “less than approximately 4.5,” which should be interpreted to include all of the above-recited values and ranges. Further, such an interpretation should apply regardless of the breadth of the range or the characteristic being described.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

What is claimed is:

1. A cannulated aspiration screw device having a longitudinal axis, wherein the cannulated aspiration screw device comprises:
(a) a shaft having a first end and a second end, wherein
(i) the first end of the shaft has a first tubular section along the longitudinal axis that is substantially cylindrical and having a first outer-diameter,
(ii) the second end of the shaft has a second tubular section along the longitudinal axis that is substantially cylindrical and having a second outer-diameter, and
(iii) the second outer-diameter is smaller than the first outer-diameter,
(b) a drilling tip section having a first end and a second end, wherein
(i) the first end of the drilling tip section is connected to the first end of the shaft and positioned along the longitudinal axis,
(ii) the second end of the drilling tip section has a drilling tip that is operable for drilling into bone,
(iii) the drilling tip comprises one or more fenestrations,
(iv) the shaft and drilling tip section are operable for receiving a removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft, and
(v) in the absence of the removable insert, the cannulated aspiration screw device is operable for flowing materials through the one or more fenestrations into the drilling tip section, from the drilling tip section to the shaft, and from the shaft to outside the shaft through the second end of the shaft.

2. The cannulated aspiration screw device of claim 1, wherein the shaft and drilling tip section comprise biocompatible transient contact only materials.

3. The cannulated aspiration screw device of claim 1, wherein the one or more fenestrations comprises at least two fenestrations.

4. The cannulated aspiration screw device of claim 1, wherein the one or more fenestrations have a diameter that is at most a 1:1 ratio of fenestration diameter to pitch.

5. The cannulated aspiration screw device of claim 1, wherein the removable insert is inserted in the cannulated aspiration screw device and the removable insert prevents the flow of materials through the one or more fenestrations, from the drilling tip section to the shaft, and out of the shaft through the second end of the shaft.

6. The cannulated aspiration screw device of claim 5, wherein the removable insert is a stylet.

7. The cannulated aspiration screw device of claim 5, wherein the removable insert seats on the one or more fenestrations.

8. The cannulated aspiration screw device of claim 5, wherein the cannulated aspiration screw device is operatively connected to a manual drill or handle.

9. The cannulated aspiration screw device of claim 1, wherein the removable insert is not inserted in the cannulated aspiration screw device.

10. The cannulated aspiration screw device of claim 9, wherein the cannulated aspiration screw device is sealably connected to an aspiration device.

11. The cannulated aspiration screw device of claim 10, wherein the cannulated aspiration screw device is sealably connected to an aspiration device with a connector.

12. The cannulated aspiration screw device of claim 11, wherein the connector is a male luer connector.

13. The cannulated aspiration screw device of claim 11, wherein the connector comprises an O-ring and a positive stop.

14. The cannulated aspiration screw device of claim 10, wherein the aspiration device is a syringe.

15. An aspiration method comprising:
(a) selecting a cannulated aspiration screw device comprising a shaft and a drilling tip section, wherein
(i) the drilling tip section comprises one or more fenestrations, and
(ii) the cannulated aspiration screw device has a removable insert that prevents materials from flowing into the cannulated aspiration screw device through the one or more fenestrations;
(b) screwing the drilling tip section of the cannulated aspiration screw device into a bone containing bone marrow;
(c) removing the removable insert from the cannulated aspiration screw device after the step of screwing the drilling tip section in the bone;
(d) connecting the cannulated aspiration screw device to an aspiration device;
(e) utilizing the aspiration device to aspirate bone marrow from the bone through the cannulated aspiration screw device;
(f) removing the cannulated aspiration screw device from the bone.

16. The aspiration method of claim 15, wherein
(a) the shaft of the cannulated aspiration screw device has a first end and a second end, wherein
(i) the first end of the shaft has a first tubular section along the longitudinal axis that is substantially cylindrical and having a first outer-diameter,
(ii) the second end of the shaft has a second tubular section along the longitudinal axis that is substantially cylindrical and having a second outer-diameter, and
(iii) the second outer-diameter is smaller than the first outer-diameter,
(b) the drilling tip section of the cannulated aspiration screw device has a first end and a second end, wherein
(i) the first end of the drilling tip section is connected to the first end of the shaft and positioned along the longitudinal axis,
(ii) the second end of the drilling tip section has a drilling tip that is operable for drilling into bone,
(iii) the shaft and drilling tip section are operable for receiving the removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft, and
(iv) in the absence of the removable insert, the cannulated aspiration screw device is operable for flowing materials through the one or more fenestrations into the drilling tip section, from the drilling tip section to the shaft, and from the shaft to outside the shaft through the second end of the shaft.

17. The aspiration method of claim 15, wherein the step of screwing the drilling tip section of the cannulated aspiration screw device into the bone containing bone marrow comprises connecting a proximate end of the shaft to a manual drill or handle and using the manual drill or handle to screw the cannulated aspiration screw device.

18. The aspiration method of claim 17, wherein the manual drill or handle is a manual drill.

19. The aspiration method of claim 17, wherein the manual drill or handle is connected to the proximate end of the shaft without any connector.

20. The aspiration method of claim 17, wherein the manual drill or handle is connected to the proximate end of the shaft with a connector that connects to both the manual drill or handle and the proximate end of the shaft.

21. The aspiration method of claim 17, wherein the step of connecting the cannulated aspiration screw device to an aspiration device comprises connecting a connector to the proximate end of the shaft and connecting the connector to the aspiration device.

22. The aspiration method of claim 21, wherein the connector is a male luer connector.

23. The aspiration method of claim 21, wherein the connector comprises an O-ring and a positive stop.

24. The aspiration method of claim 15, wherein the aspiration device is a syringe.

25. The aspiration method of claim 15 further comprising removing the connector from the cannulated aspiration screw device.

26. The aspiration method of claim 15, wherein the step of removing the cannulated aspiration screw device from the bone comprises connecting a proximate end of the shaft to a manual drill or handle and using the manual drill or handle to unscrew the cannulated aspiration screw device.

27. A kit comprising a plurality of components, wherein the plurality of components comprise:
(a) a cannulated aspiration screw device comprising a proximate end and a distal end, wherein
(i) the proximate end of the cannulated aspiration screw device comprises a shaft,
(ii) the distal end of the cannulated aspiration screw device comprises a drilling tip section,
(iii) the drilling tip section comprises one or more fenestrations, and
(iv) the cannulated aspiration screw device is operable to be screwed into bone using the drilling tip section;
(b) a removable insert that is operable to be inserted into the proximate end of the cannulated aspiration screw device and through the shaft and drilling tip section to prevents materials from flowing into the cannulated aspiration screw device through the one or more fenestrations; and
(c) a connector that is operable to sealably connect the proximate end of the cannulated aspiration screw device to an aspiration device.

28. The kit of claim 27, wherein
(a) the shaft of the cannulated aspiration screw device has a first end and a second end, wherein
(ii) the first end of the shaft has a first tubular section along the longitudinal axis that is substantially cylindrical and having a first outer-diameter,
(ii) the second end of the shaft has a second tubular section along the longitudinal axis that is substantially cylindrical and having a second outer-diameter, and
(iii) the second outer-diameter is smaller than the first outer-diameter,
(b) the drilling tip section of the cannulated aspiration screw device has a first end and a second end, wherein
(i) the first end of the drilling tip section is connected to the first end of the shaft and positioned along the longitudinal axis,
(ii) the second end of the drilling tip section has a drilling tip that is operable for drilling into bone,
(iii) the shaft and drilling tip section are operable for receiving the removable insert through the second end of the shaft, through the first end of the shaft, and to the one or more fenestrations in the drilling tip section to prevent the flow of materials through the one or more fenestrations, from the drilling tip section to the shaft, and out of the shaft through the second end of the shaft, and
(iv) in the absence of the removable insert, the cannulated aspiration screw device is operable for flowing materials through the one or more fenestrations into the drilling tip section, from the drilling tip section to the shaft, and from the shaft to outside the shaft through the second end of the shaft.

29. The kit of claim 27, wherein the aspiration device is a surgical syringe.

30. The kit of claim 27, wherein the plurality of components further comprises the aspiration device.
